(19) United States
(12) Patent Application Publication
(10) Pub. No.: US 2026/0199539 A1
(43) Pub. Date: Jul. 16, 2026

(54) Electrically-Polarized Surfaces Generate Reactive Oxygenated Species for Fast Deactivation of Broad-Band Microorganisms

(71) Applicant: Board of Trustees of Southern Illinois University, Springfield, IL (US)

(72) Inventors: Punit Kohli, Carbondale, IL (US); Annie Yojaira Vargas Lizarazo, Carbondale, IL (US); Md Aswad Ali, Carbondale, IL (US); Nehal Ahmed Mazumder, Carbondale, IL (US); Scott Hamilton-Brehm, Murphysboro, IL (US)

(21) Appl. No.: 19/088,200

(22) Filed: Mar. 24, 2025

Related U.S. Application Data
(60) Provisional application No. 63/569,194, filed on Mar. 24, 2024.

(51) Int. Cl. A61L 2/208; A61L 2/035; A61L 2/232; A61L 9/04; A61L 103/50

(52) U.S. Cl. CPC A61L 2/208; A61L 2/035; A61L 2/232; A61L 9/046; A61L 2103/50; A61L 2202/11; A61L 2209/211

(57) ABSTRACT

A device with microbiocidal or microbiostatic properties that is provided with a device body that contains a first external surface and a second external surface that resides opposite the first external surface. The device is further provided with an at least one antimicrobial metal component in electrical relationship to said first external surface and said second external surface.

### GOVERNMENT SUPPORT CLAUSE

No federal funding was provided in support of this invention.

### FIELD OF THE INVENTION

The present invention relates to the prevention of infection and, more particularly, to the provision of methods and devices for inhibiting exposure to microbes and infection.

### BACKGROUND OF THE INVENTION

Owing to the recent outbreaks of infectious diseases of SARS-CoV-2 (2019-up to date)¹, Monkeypox (2022- up to date)², Influenza A (a widespread yearly outbreak)³, measles morbillivirus (2017-2019)⁴ etc., and related emerging antimicrobial resistance diseases^{5, 6}, the design and development of broad-band antimicrobial surfaces and materials is considered the new “silver bullet” strategy to reduce the fast spread of contagious diseases⁷⁻¹⁰. Recently, a great number of antimicrobial approaches including the use of biological biocides (e.g., antimicrobial peptides^{7, 8, 11, 12}, enzymes^{7, 8, 12}, bacteriophages^{7, 8}, etc.), organic biocides (e.g., antibiotics^{5, 7, 8, 10, 12}, small molecules inhibitors^{7, 13, 14}, organic cationic and non-cationic compounds^{7, 8}, high molecular weight polymers^{7, 15}, etc.) and metal biocides (e.g., Ag^{7, 9, 16-19}, Cu^{7, 14, 18}, MgO²⁰, Fe₂O₃²¹, TiO₂²², CuO^{7, 14, 23}, CeO₂²⁴, ZnO^{7, 23, 24, 25}) have been reported.

At least one prior art from Mihut et al. suggests that copper or silver coatings may be used to create an antimicrobial effect. However, there are deficiencies in using a strict elemental (non-ionic) monometallic metallic coating as this effort has a limited effect in killing micros. Another important deficiency of Mihut et al. is the requirement of high electric current. The electric current required is many amperes which is extremely high, and thus dangerous. This poses severe safety concerns, and results in more power consumption, and heat generation.

Biological biocides are not commonly used in the field due to the high-cost production and low stability to challenging environments (pH, temperature, radiation, etc.), low selectivity and non-rechargeable or regenerative surfaces¹². Organic biocides are molecules synthetized at large scale embedded, supported or encapsulated in organic matrix (polymers)^{7, 12} whereby the deactivation or killing mechanism is based on the biocide diffusion to the host cell membrane followed by exposure to the host cell content^{7, 12}. This approach is considered ecofriendly because the degradation of the matrix is nontoxic^{7, 12}. However, similar to biological biocides, these matrices are non-rechargeable, and easily contaminated with the host cell content reducing the lifetime and efficiency of the matrix^{7, 12}.

Metal biocides are the most well-known approach due to the broad range of applications, adaptability to different matrices/surfaces, and simple to moderate synthesis and deposition methods^{7, 9, 10, 12, 26, 27}. The popularity of this method is based on the versatile antimicrobial features of the metal and metal oxides particles: generation of oxidative stress generated on the cell^{9, 21, 26, 28}, cell toxicity due to ion metal release^{7, 12, 16, 26}, and cell membrane damage in absence of oxidative stress^{20, 27}. Even though the antimicrobial features mentioned damage the cell, the key step for the cell deactivation for many antimicrobial surfaces and materials is the production of reactive oxidant species (ROS) and reactive chlorinated species (RCS) on the metal surface suppressing the antioxidant response of the host cell^{7, 16, 21, 26}. ROS not only regulate cellular reproduction, but also play a crucial role in the cell defense mechanism^{29, 30}. Depending on the ROS and RCS concentration, the host cell cycle could be affected, inhibited, and even activate the cell death process^{29, 30, 31}. ROS are produced by cells, and chemically generated—through photocatalysis⁷⁻⁹, light activated molecules³², light induce³³, etc., —as an oxygen derived intermediate of water and oxygen redox reactions^{17, 28}. Through the chemical method, four major ROS species are produced that shows high toxicity for host cells: Superoxide anion radical (O₂^{•-}), hydroxyl radical (OH^{•}), singlet oxygen (¹O₂) and hydrogen peroxide (H₂O₂)^{17, 28}.

### SUMMARY OF THE INVENTION

Devices and methods are provided for inhibiting exposure to microbes and infection according to the present invention. The device provided exhibits enhanced antimicrobial activity by utilizing a specific design and metal composition that generates an increased amount of antimicrobial ROS and RSC upon application of an electrical current to the device. The present device also requires much less current than Mihut et al. (two to three orders of magnitude less). This provides a much safer process, and results in less power consumption and heat generation.

The present invention is generally a device that may be described as an antimicrobial device. In one embodiment, the antimicrobial device includes a device body containing a first external surface, a second external surface, and an at least one antimicrobial metal component in an electrical relationship to the first external surface and the second external surface. An electrical power source is optionally provided connected to a first electrically conductive lead and a second electrically conductive lead such that the first electrically conductive lead and the second electrically conductive lead are electrically connected so that current flows from the first electrically conductive lead to the second electrically conductive lead through the device body causing the production of ROS and RCS.

The device may be any object used in everyday life, including those specifically identified hereinbelow. One means of doing this such that high amperes are not required involves using salts of the metal ions such as Cu2+ or Cu+. The utility of these salts requires less current to achieve a more effective and efficient bacterial ablation or deactivation response.

The present invention also provides a method or methods for inhibiting exposure to microbes, infection, and proliferation of those. One method includes the steps of providing a device body with an antimicrobial metal component on, within or on and within the device body to an environment and applying an electrical current to the antimicrobial metal component to generate Reactive Oxygen Species or Reactive Chlorinated Species.

### DESCRIPTION OF THE DRAWINGS

FIG. 1 shows a schematic of the device whereby the device body containing the electrically conductive elements (Cu²⁺ and Ag) is shown in purple and dark grey (now in grayscale), while the first electrically conductive lead and second electrically conductive lead (containing Ag in this case) are shown in light grey.

FIG. 2A shows a schematic of the deactivation of microorganisms and lentivirus with the ENM) device treatment. An aqueous droplet (large light blue (now clear) sphere) containing three perforated microorganisms (rose, green, and pistachio which have been converted to grayscale) was placed on an ENM device (grey plate) that was electrically-polarized with a low-power battery. The (+) and (−) represent + and − polarities of a battery respectively. The smaller cyan, red, and light pink (now converted to grayscale) spheres inside of the large light blue sphere represent ^{•}OH, H₂O₂, and HOCl respectively.

FIG. 2B shows a schematic of the ENM fabrication procedure, where a substrate (white) was first deposited with Cu(II) (blue (now grayscale) particles) by immersion in a 0.1 M CuSO₄ solution, which was followed by a ~90 nm silver coating (dark grey).

FIG. 2C shows an optical image of a typical ENM device.

FIG. 2D shows the energy dispersive spectroscopy (EDS) mapping of an ENM device. The elements Cu and Ag are shown in blue and purple (now shown in grayscale) colors respectively. Element Cl is not shown in the elemental map.

FIG. 2E shows an EDS energy spectrum of ENM reveals the K_{α} peaks of elements C, O, S, Cl, and Ag at 0.277 keV, 0.525 keV, 2.307 keV, 2.621 keV and 2.984 keV respectively, whereas Cu peaks appeared at 0.930 keV (L_{α}) and 8.040 keV (K_{α}).

FIG. 2F shows a fluorescence micrograph of three 40 μM fluorescein-containing water droplets deposited on an ENM_{3V} for τₐₚₚ=10 min, exhibiting θ=115±6° (n=3). The scale bar is 5 mm.

FIG. 2G shows an atomic force micrograph (AFM) of a silver step deposited on a glass slide for the measurement of silver thickness coating.

FIG. 2H shows a line profile of the silver step yielded silver thickness of ≈93 nm (scan size=20 m×20 μm).

FIG. 2I shows a high-resolution AFM image of the ENM surface exhibited an average silver particle size of 29±5 nm (n=172).

FIG. 2J shows a histogram of the Ag particle size on the ENM device shown in FIG. 2I.

FIG. 3 shows the fabrication of the device where in this example, the surface was soaked into a 0.1 M CuSO₄ solution for 1 hour and subsequently dried at room temperature. Each dried surface was placed flat on a glass slide and Ag was sputtered for 6 minutes on each side. The electrically conductive leads (grey strips) were placed on vertically opposite side of the Ag-coated device body.

FIG. 4A shows the concentration of copper in the Cu²⁺—in the device body and UV-Vis absorption curves of CuSO₄·5H₂O for the estimation of molar absorption coefficient.

FIG. 4B shows how the value ε=11.9 L mol−1 cm⁻¹ at 800 nm was obtained for the aqueous solution of CuSO₄·5H₂O.

FIG. 4C shows at typical UV-Vis absorption curves of the Cu(II) released from the Cu(II)-coated surfaces after they were washed with water. Release of Cu(II) in solution is shown in the spectra: after the first washing cycle (1^{st} washing), second washing cycle (2^{nd} washing) and third washing cycle (3^{rd} washing). The ENM device area was 144 cm². The average [Cu(II)]-washing cycles yielded a total Cu(II) concentration of 15.7 ppm cm⁻² (n=4). In comparison, the ICP-MS analysis for copper and silver released from the ENM3V devices showed 125 ppb cm⁻² and 0.45 ppm cm⁻², respectively (τₐₚₚ=30 min). A much lower Cu and Ag elemental concentrations in the ICP-MS data than UV-Vis experiments is attributed to low τₐₚₚ treatment times for ICP-MS experiments. These experiments, however, provide metallic elemental concentrations in the solutions that are relevant to microorganisms and lentivirus deactivation experiments.

FIG. 5A shows the thickness of Ag coating on the device body.

FIG. 5B shows the atomic force microscopy (AFM) image of the Au/Pd coating on a glass substrate, where the sputtering time was 3 min at 50 mTorr pressure using 40 mA current.

FIG. 5C shows the resulting thickness of Au/Pd coating.

FIG. 6A shows viable cells (CFU/mL) of control and after treated with a mask layer coated with silver copper (MAC) under 0V and 3V applied from three different kingdoms: bacteria, fungus, and virus. (A) Cell viability and effectiveness (left y-axis) of S. aureus, A. Baumannii, and P. aeruginosa of control and MAC treated at 0V and 3V for 5 minutes. The control was the same materials as MAC but with any coating on it.

FIG. 6B shows viability and effectiveness of a mixture of Siuc 2 and Siuc 11 of control and MAC treated at 0V and 3V for 5 minutes. There are three gram-negative (A. Baumannii, P. Aeruginosa, and Siuc 2) and two gram-positive (S. aureus and Siuc 11) in the treated groups.

FIG. 6C shows a comparison of MAC treated (0V and 3V) for Aspergillus fumigatus for 10 and 20 min.

FIG. 6D shows viability of lentivirus virus treated with MA (mask layer coated with silver) for 10 and 20 min at 0 and 3 V. For all experiments, results were presented as the mean standard deviation of three independent experiments for statistical analysis (n≥3). Cell viability and effectiveness are drawn on left and right axes respectively. Kruskal Wallis (ANOVA) was performed to analyze the data at p<0.05 (statistically significant). The initial concentration (CO) of S. aureus, A. Baumannii, and P. aeruginosa, SIUC-2 and SIUC-11 prior to treatments were 3×106, 1.5×106, 2.8×106, and ~104 respectively.

FIG. 7A shows the metal coatings of copper and silver were applied to three-dimensional microparticles composed of silicon oxide, silanol groups, and carbon atoms homogenously distributed.

FIG. 7B shows an EDS color map of the distribution of copper (green converted now to grayscale)) in the three-dimensional microparticle (blue converted now to grayscale)).

FIG. 7C shows EDS elemental mapping of the copper-decorated microparticles with the characteristic peak at 0.93 keV and 8.04 keV and silicon at 1.73 eV.

FIG. 7D shows an optical top image of a device containing three-dimensional microparticles decorated with silver and copper.

FIG. 8 shows a coating comprising metallic nanowires as the active materials along with a thin nanoscale Ag coating on a device body composed of an air filter, paper, glass and textile fabric surfaces.

FIG. 9 shows the Aquadage E, copper salt, graphite powder, polymer and liquid silicate components being combined and applied to a control surface to produce a conductive graphite surface.

FIG. 10 shows the redox reaction pathways that generated ROS and RCS through the device. Redox reaction pathways which generated ROS and RCS in ENM-treated solutions. The figure contains two parts: triangle (top) and square (bottom) redox cycles. The top triangle provides possible Cu(0/I/II) redox reactions occurring in the solutions treated with ENM devices. The square provides various possible reactions that generate ROS and RCS in the system. The size of the circle in the square represents the expected/measured concentration of the species. (ROS) and (RCS) colored species are reactive to cells, whereas grey colored rectangles are less reactive/non-toxic to cells. For some reactions half-cell reduction potential (in volts) and second-order rate constants (in M⁻¹s⁻¹) are also provided. The dotted circles in the rectangular part represent species that are either not detected or are formed at concentrations which we were not able to detect. The solid and dashed arrows represent thermodynamically accessible and thermodynamically inaccessible/less accessible reactions respectively. The external power is represented by a yellow circle with e⁻. H₂O₂ can be generated by two successive one-electron reduction mechanism or a single two-electron O₂ reduction. The later mechanism is denoted by *. Some reactions depicted in the figure can occur through multiple pathways; but not all possible reaction pathways are shown in the figure.

FIG. 11 shows a schematic of Cu(I) diffusion into the bulk solution and formation of ROS and RCS in the bulk solution. The large hemispheres represent aqueous droplets ranging from many 100s of micrometers to ~ centimeter in dimension. (1) Electrochemical and electrically-induced reduction of Cu(II) to Cu(I) on the ENM electrode. (2) O₂ diffuses to the ENM-water interface, whereas Cu(I) can diffuse away from the ENM-water interface. (3) O₂ is reduced to O₂^{•-}. (4) Cu(I)-mediated reduction of O₂° produces H₂O₂ (light blue sphere) in the bulk solution. (3′) Two-electron O₂ reduction produces H₂O₂ utilizing two Cu(I) species. (5) A Fenton-like reaction between H₂O₂ and Cu(I) produces ^{•}OH. (6) Reaction of H₂O₂ in the presence of Cu(II) and Cl⁻ produces HOCl. ^{•}OH and HOCl can react with microorganisms and lentivirus, killing or deactivating them. Hypothetical scale bars are provided for visual aid to dimensions of the water droplets. Ions, radicals and other molecular species are not drawn to the scale.

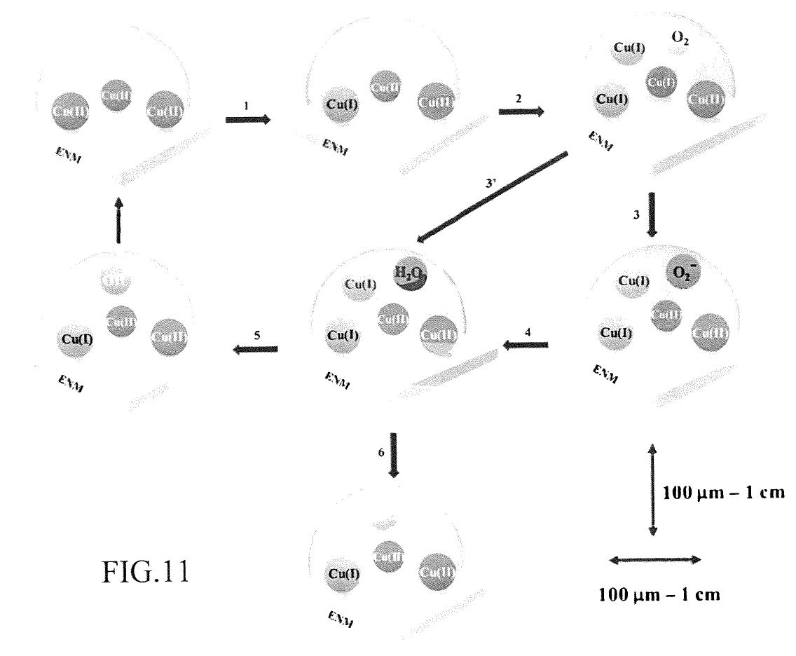

### DETAILED DESCRIPTION OF THE PREFERRED EMBODIMENTS

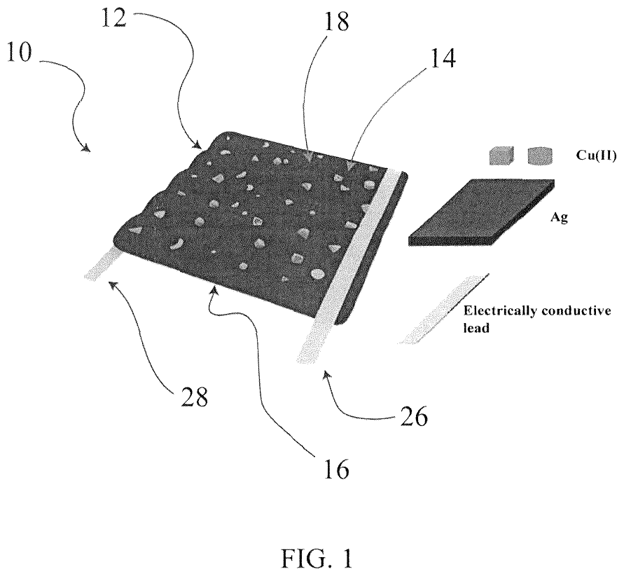

Referring to FIG. 1, a device 10 for inhibiting exposure to microbes and infection is presented as follows: a device body 12 containing a first external surface 14, a second external surface 16, and an antimicrobial metal component (which could be one or more metals that have antimicrobial properties) 18, a first electrically conductive lead 26 being attached to the first external surface 14 of the device body 12, a second electrically conductive lead 28 being attached to the second external surface 16 of the device body 12 and being electrically isolated from the first electrically conductive lead 26, and an electrical power source (not shown, and may be referred to as just “power source”) connected to the first electrically conductive lead 26 and the second electrically conductive lead 28 such that the first electrically conductive lead 26 and second electrically conductive lead 28 are electrically connected so that current flows from the first electrically conductive lead 26 to the second electrically conductive lead 28 through the device body 12 causing the production of ROS and RCS at the antimicrobial metal component 18. The electrical power source may provided by an internal battery, a capacitor or other AC or DC power source.

The first electrically conductive lead 26 and second electrically conductive lead 28 are electrically insulated from each other through the device body 12. The device body 12 accommodates current flow from the first electrically conductive lead 26 to the second electrically conductive lead 28.

A power source powers the device 10 according to the present invention. Such a power source may be any suitable power source such as a battery, capacitor, electrochemical cell, solar cell or power outlet One terminal of the power source is in electrical communication with the first electrically conductive lead 26. The second terminal of the power source is in electrical communication with at least the second electrically conductive lead 28.

A metal component includes an antimicrobial metal. An antimicrobial metal is one which inhibits one or more microbes or other organism, such as bacteria, protozoa, viruses, and fungi. An antimicrobial metal may be microbiocidal or microbiostatic.

The metal component contains an amount of the antimicrobial metal, the amount in the range of 1%-100% by weight of the total composition of the metal component, although in particular embodiments, lower amounts may be included. In general, the metal component included in the device 10 contains an amount of antimicrobial metal in the range of about 1 nanogram to about 1 kilogram. The metal component preferably contains at least 50 percent by weight of the antimicrobial metal, further preferably contains at least 75 percent by weight of the antimicrobial metal and still further preferably contains at least 95 percent by weight of the antimicrobial metal. In another preferred embodiment, the metal component is substantially all antimicrobial metal.

The metal component may be provided in any of various forms, illustratively including, a substantially pure metal, an alloy, a composite, a mixture, and a metal colloid. Thus, in one embodiment, a metal or non-metal component is a substance doped with the antimicrobial metal. For instance, in a particular example, a stainless steel and/or titanium alloy including an antimicrobial metal may be included in a metal component.

Antimicrobial metals include transition metals and metals in columns 10-14 of the periodic table. Such metals illustratively include silver, gold, iron, zinc, copper, cadmium, cobalt, nickel, platinum, palladium, manganese, and chromium. In certain embodiments, lead and/or mercury may be included in amounts not significantly toxic to a user.

By example, the antimicrobial properties of silver are particularly well-characterized and the metal component preferably contains an amount of silver, the amount in the range of 0.1 percent to 100 percent by weight of the total composition of the metal component, although lower amounts may be included in particular embodiments. The metal component preferably contains at least 50 percent by weight of silver, further preferably contains at least 75 percent by weight silver and still further preferably contains at least 95 percent by weight silver. In another preferred embodiment, the metal component is substantially all silver.

Materials other than an antimicrobial metal may also be included in the metal component. For instance, the metal component may further include metals which are non-antimicrobial in one configuration according to the invention. To illustrate this concept, the metal component may be used to provide structural support and lower cost of the metal component. In an alternative embodiment, a non-metal constituent is included in the metal component. For instance, the non-metal constituent is used to provide structural support and lower cost of the metal component. Exemplary non-metal constituents include such substances as inorganic and organic polymers, and biodegradable materials. A non-metal constituent or non-antimicrobial metal included in a metal component may be biocompatible. Preferably, the metal component is electrically conductive.

Copper is also a preferred metal included in a metal component and a metal component preferably contains an amount of copper in the range of 0.1%-99% by weight of the total composition of the metal component, although lower amounts may be included in particular embodiments. Silver is also a preferred metal included in a metal component and a metal component preferably contains an amount of silver in the range of 0.1%-99% by weight of the total composition of the metal component, although lower amounts may be included in particular embodiments. In one embodiment, at least 0.1% by weight copper is included. In another preferred embodiment, the metal component is 99% copper. In one embodiment, at least 0.1% by weight silver is included. The metal component contains at most 100% by weight silver. However, in a most preferred embodiment, the metal component contains at least 95% by weight silver. The metal components that comprise of the antimicrobial metal components may be applied in a layer to the device body 12 by absorption, coating and electroless deposition of the metal. The non-metal component in the form of a coating ranges in thickness between 0.1×10⁻⁹ m and 5×10⁻⁶ m.

A combination of at least two metals is preferred as included in the metal component as a coating in contrast to a mono-metallic coating. In some instances, certain metals may be more effective at inhibiting growth and/or killing particular species or types of bacteria. For example, particular metals are more effective at inhibiting growth and/or killing Gram positive bacteria, while other metals are more effective against Gram negative bacteria as exemplified in the examples described herein.

In a particular embodiment, both silver and copper are included in a metal component. A combination of silver and copper may provide a synergistic antimicrobial effect. For instance, a lesser amount of each individual metal may be needed when a combination is used. Additionally, a shorter time during which the device is activated may be indicated where a synergistic effect is observed, allowing for conservation of a power source. The ratio of copper to silver in a metal component may range from 1,000:1-1:1,000. In one embodiment, a metal component preferably contains an amount of a copper/silver combination in the range of 1-100 percent by weight of the total composition of the metal component, although lower amounts may be included in particular embodiments. In another embodiment, at least 50 percent by weight of a copper/silver combination is included, further preferably a metal component contains at least 75 percent by weight of a copper/silver combination and still further preferably contains at least 95 percent by weight of a copper and silver in combination. In another preferred embodiment, the metal component is substantially all copper and silver.

In a preferred embodiment, the metal component is in the form of a coating disposed on the external surface of the device body 12. The coating can be applied by any of various methods illustratively including dunk coating, thin film deposition, sputter coating, vapor deposition, or electroplating. The metal component in the form of a coating ranges in thickness between 0.1×10⁻⁹ meters to 5×10⁻⁶ meters, inclusive, preferably 1×10⁻⁷ meters to 5×10⁻⁵ meters, inclusive, and more preferably between 1×10⁻⁷ meters to 5×10⁻⁶ meters in thickness.

In an additional embodiment, an alternative for metal coating, graphite from a pencil provides an electrically conductive layer. The non-metal component in the form of a coating ranges in thickness between 0.1×10⁻⁹ m to 5×10⁻⁶ m. The copper amount varies in the range of 10⁻⁵%-99% by weight of the total composition of the device 10. The device 10 is able to deactivate bacteria from 10¹ to 10¹⁰ cell forming units mL⁻¹ in a period of 5 min to 2 hours. The required potential applied varies from 0 V to 9 V.

Optionally included in electrical communication with the device 10 according to the present invention is circuitry adapted to modulate a current from the power source. For example, a resistor, a switch, a signal receiver, a relay, a signal transmitter, transformer, a sensor, or a combination of these or other such components and connectors may be included. This circuitry may optionally be configured as a circuit board arrangement. In a preferred embodiment, all or part of the circuitry adapted to modulate an electrical current is housed in a cavity in one or more portions of the device 10.

It is appreciated that, in the context of preferred embodiments of the device 10 or system according to the present invention including at least two electrically conductive leads of the device 10, wherein the electrically conductive leads are electrically isolated by the device body 12. The device body 12 contains the metal component which may be in the form of a metal-containing coating. In this context, the metal-containing coating on the one or more elements of the device is preferably present on at least 50 percent of the external surface of one or both elements of the device. More preferably the metal-containing coating on the one or more elements of the device is preferably present on at least 75 percent of the external surface of one or both elements of the device, and further preferably the metal-containing coating on the one or more elements of the device is preferably present on substantially all of the external surface of the one or more elements of the device 10.

The metal coating may be disposed on the surface of the device body 12 in a patterned fashion. For example, interlocking stripes of a metal component and a device body 12 may be arranged on the surface of a device. Such a pattern is preferably designed to inhibit microbes in a continuous region on the device.

A metal coating on element is preferably disposed on an external surface as a single continuous expanse of the device body 12 material.

An electrically conductive lead may be in the form of a wire, paint, ribbon, or foil disposed on the external surface of a device. Such an electrically conductive lead may be attached to the device body 12 by soldering, welding, by an adhesive, or the like.

In an alternative embodiment, a device 10 made of a material including an antimicrobial metal may be formulated such that the antimicrobial metal is distributed non-uniformly throughout the device body 12. For instance, the antimicrobial metal may be localized such that a greater proportion of the antimicrobial metal is found at or near one or more surfaces of the device body 12.

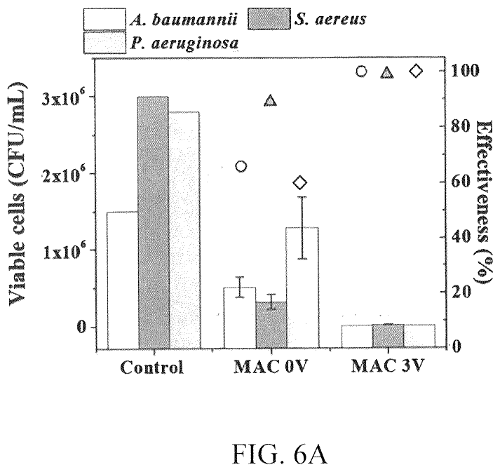

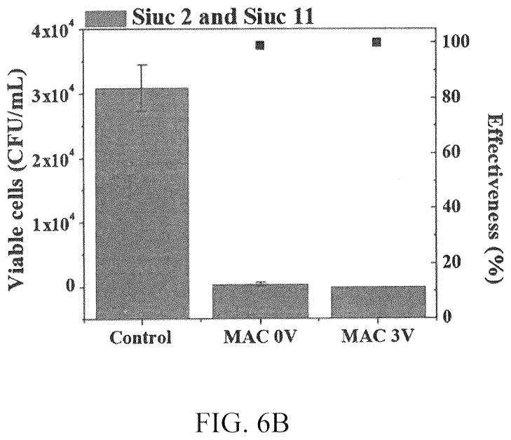

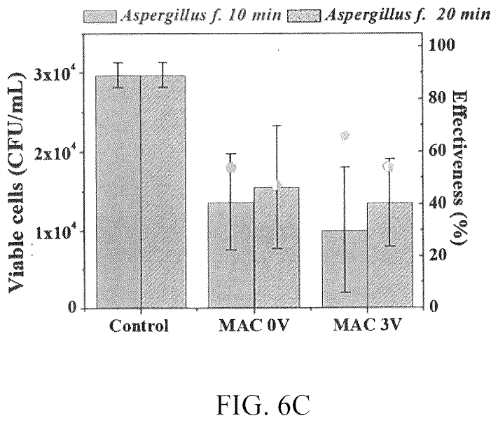

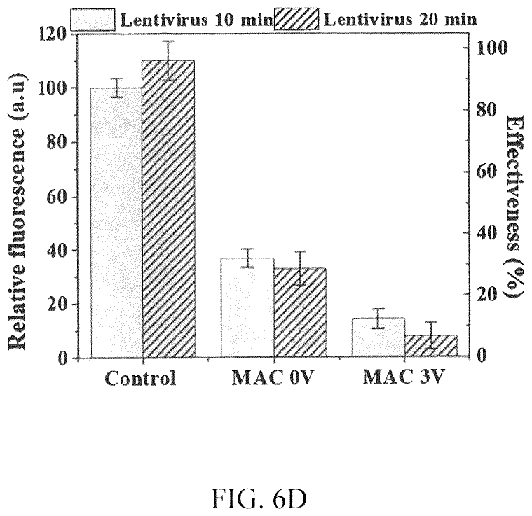

An important benefit with the present device 10 is the reduced electric current flowing through the systems. Prior systems require one to three orders of magnitude higher current than the present device. Prior systems that utilize higher current thus could pose safety concerns and result in more power consumption and heat generation. Now referring to FIG. 6 reduction of viable cells (CFU/mL) represented by three different kingdoms: bacteria, fungus, and virus is observed after treated with a mask layer coated with silver and copper (MAC) under applied voltages of 0V and 3V. In FIG. 6A substantial reduction of cell viability was observed for the bacterial species S. aureus, A. baumannii, and P. aeruginosa when treated at 0V and 3V for 5 minutes. In FIG. 6B substantial reduction of cell viability was observed for the bacterial species Siuc 2 and Siuc 11 when treated at 0V and 3V for 5 minutes. In FIG. 6C substantial reduction of cell viability was observed for two species of Aspergillus fungi when treated at 0V and 3V for In FIG. 6D substantial reduction of viability was observed for lentivirus when treated at 0V and 3V for 10 and 20 min.

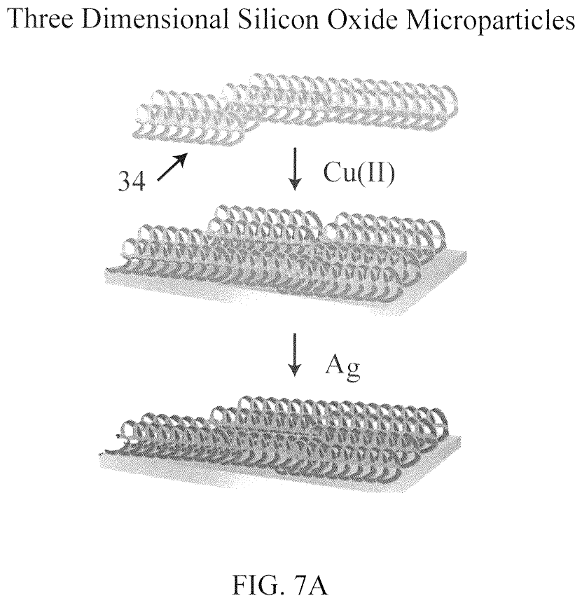

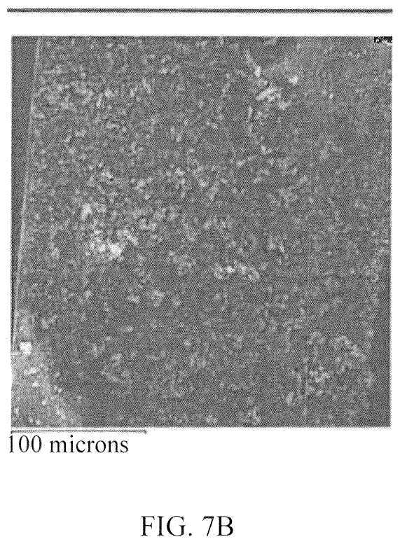

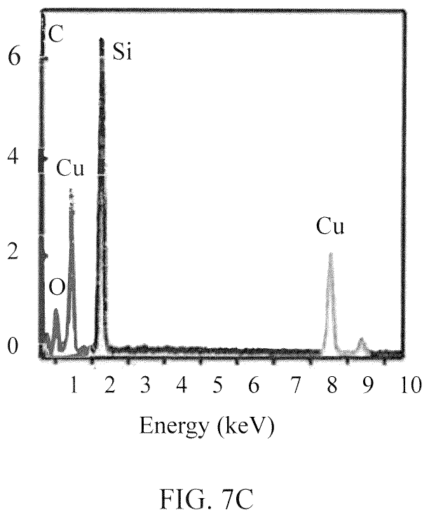

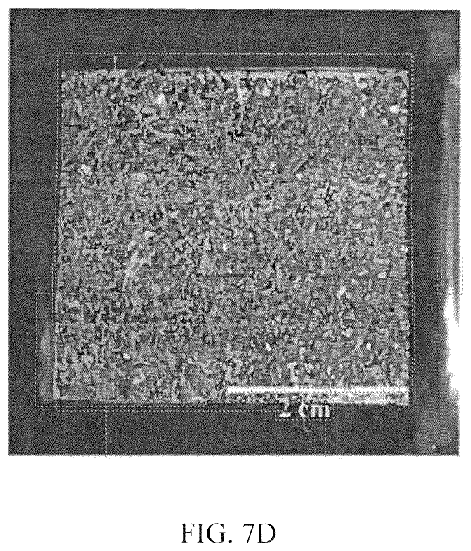

Now referring to FIG. 7 and more particularly, FIG. 7A, as an example of previous embodiment, the metal coatings of copper and silver were applied to three-dimensional microparticles 34 composed of silicon oxide, silanol groups, and carbon atoms homogenously distributed (FIG. 7). These microparticles are obtained after the anisotropic thermal oxidation of polydimethylsiloxane (PDMS) thin films on glass substrate.

The use of three-dimensional microparticles 34 provide enhanced stability of the metal coating along with increased hydrophobicity. The three-dimensional microparticles 34 size can vary in a range of 10 μm to 5000 μm in width, 0.1 mm to 10 cm in length, and a thickness of 10 nm to 3000 μm. The size of devices 10 employing the three-dimensional microparticles 34 varies from 0.1 mm×0.1 mm to 1 μm×1 μm. The weight of the devices 10 containing three-dimensional microparticles 34 vary from 1 mg to 500 g. Three-dimensional microparticles 34 are created by using polymer microparticles or other non-metal microparticles. The polymer microparticles or other non-metal microparticles are coated with various metals including copper, silver, gold, titanium, chromium, iron, nickel, zinc, palladium, platinum, ruthenium, iridium, tungsten, cobalt, vanadium, manganese, molybdenum, and other metals by absorption, electroplating, magnetron and non-magnetron sputtering, electroless and other deposition techniques. In one example, the weight of copper and silver on three-dimensional microparticles 34 varies in the range of 0.1-99% by weight of the total composition of the device [from 1 μg/mL to 1000 μg/mL of the metal deposited in the microparticles]. The metals in one example were deposited on three-dimensional polymer microparticles by electroless and plasma sputtering deposition. The thickness of copper and silver varied between 0.1×10−9 m to 5×10−6 m. This device exhibited microorganisms (SIUC 2, SIUC 11, E. coli, Candida albicans, Aspergillus fumigatus, and Fusarium virguliforme) deactivation in a range of 101 to 1010 cell forming units mL−1 in a period of 1 min to 24 h with an applied potential per centimeter square of the device of 0 V per cm2 to 10 V per cm2 (V per cm2 represent applied potential in volt divided by the area of the device). The deactivation of microorganisms by treatment with the coatings can happened through direct contact and indirect contact through aqueous environment. The distance between microbes and coatings can range from 0.1 nm to many meters (E.g. 10 meters). These coating particles are effective in deactivating microorganisms present on aerosols, water droplets, and gaseous/vapor conditions. This coating of microparticles can be applied to air and water filters, masks, textiles, filters for water decontamination, door and window knobs/handles, toilet seats and handles, equipment present in schools, university, hospital, industry, railway station, airport, agriculture, ships, airplanes, submarines, and homes. The use of three-dimensional microparticles 34 may also be applied to live plant pots to protect seeds and plants against bacteria, fungi, viruses, and archaea.

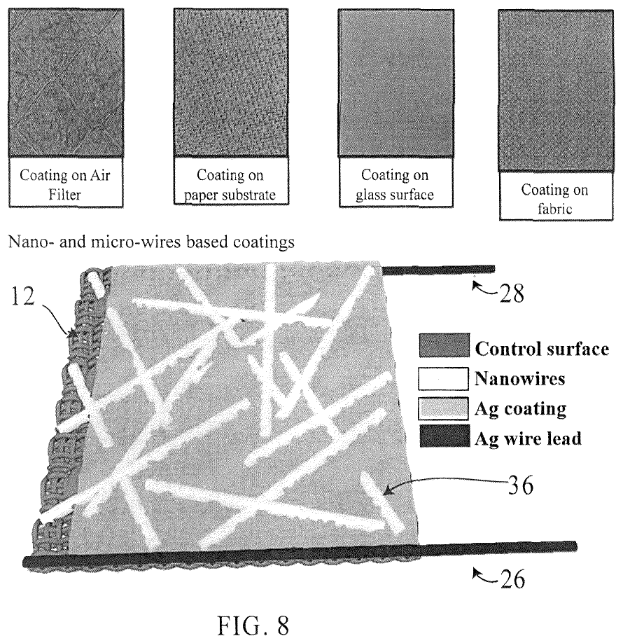

Now referring to FIG. 8, in an alternative embodiment, a coating comprising monometallic and bimetallic nanowires 36 as the active materials along with a thin nanoscale Ag coating on a device body 12 composed of textile fabric. (FIG. 8) The nanowires 36 may be composed of monometallic or multimetallic nanowires 36 (copper, silver, gold, copper, titanium, chromium, iron, nickel, zinc, palladium, platinum, tungsten, cobalt, vanadium and other metals). Preferably, monometallic Cu, Ag, Fe, and the mixture of a combination of any of these nanowires 36 or bimetallic Cu—Ag nanowires 36, and a mixture of monometallic Cu nanowire and Fe nanoparticles were also employed. The length of the nanowires 36 ranges from 10 nm to 1 mm, whereas the diameter of the ranges from 1 nm-5000 μm. Weight of metal of nano- and micro-wires per square centimeter of the device is 0.01 mg cm⁻²-100 mg cm⁻². The distribution of the nanowires 36 on the surfaces ranges from 10⁻³ μgcm² to 100 mgcm⁻². The ratio of metal nanowires 36 in the mixtures of a combination of nanowire can be 0-100. The nanoscale Ag coating can be realized by electroless, electroplating, or sputter coating instrument including plasma, magnetron etc. The thickness of the nanoscale Ag coating ranges from 0.1 nm-5 mm. The size of these device varies from 0.1 mm×0.1 mm to 1 m×1 m. The microbes deactivated with these devices includes SIUC2, SIUC 11 and E. coli by treating them with these devices for 1 minute to 24 hours at an applied potential in air/gaseous environment: of 0 V cm⁻²-10 V cm⁻². The distance between microbes and coatings can range from 0.1 nm to many meters. The bacteria concentration was 0 to 10¹⁰ cells forming units per mL. Potential applications are air and water filters, masks, textiles, filters for water decontamination, door and window knobs/handles, toilet seats and handles, equipment present in schools, university, hospital, industry, railway station, airport, agriculture, ships, airplanes, submarines, and homes, and in live plant pots to protect seeds and plants against bacteria, fungi, viruses, and archaea located in air, gaseous or and ambient environments.

In another embodiment, device 10 is composed by copper and silver can be applied in antimicrobial decontamination of water. Device 10 in a range of 1 cm×1 cm to 1 m×1 m can be used to decontaminate water sources of volume ranging from 10 mL to 10 L containing 1×10¹ cells mL⁻¹-10×10¹⁰ cells mL⁻¹ through a treatment with a period of 30 mins to 10 hours with an applied potential of 1 V-11 V range.

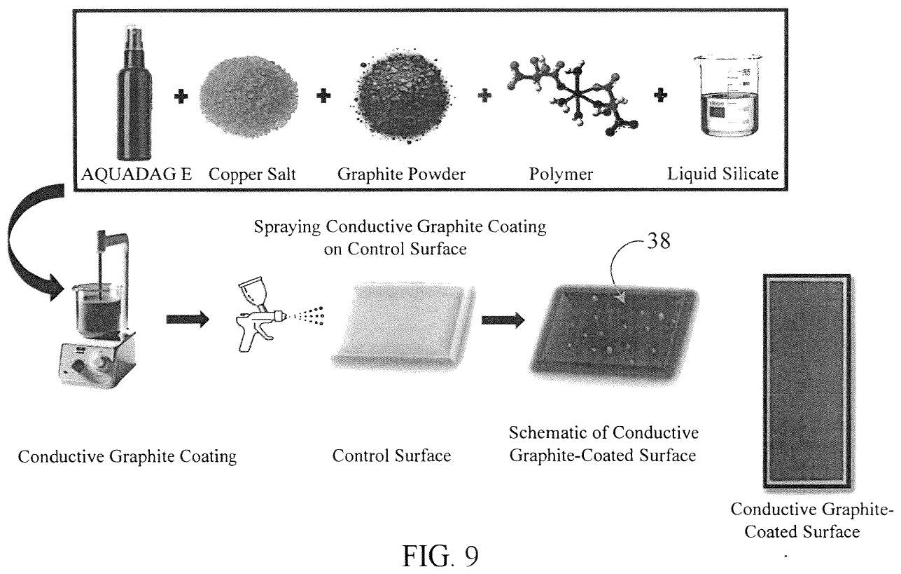

Now referring to FIG. 9, in another embodiment, a conductive graphite solution or graphite coating was applied on various surfaces to create a conductive graphite coating 38. One set of surfaces may include flat, non-planar, and curved surfaces composed of glass, brass and bronze doorknobs. Another set of surfaces may be plastic flexible surfaces may be composed of polypropylene, polyethylene, polyester etc. And another set of surfaces may be comprised of cellulose, cellulose/mineral, cellulose/polymer/plastics papers, and polymer (plastics) filters. In one embodiment, the graphite solution is an aqueous dispersion composed of liquid graphite (AQUADAG E) (1-99%, weight by weight), graphite powder (1-99%, weight by weight), metal salt (0.0001-99%, weight by weight), liquid silicate (1-99%, weight by weight), and polymers (polyvinylpyrrolidone, potassium sodium tartrate) (0.1-99%, weight by weight). Salts of copper, silver, gold, titanium, chromium, iron, nickel, zinc, palladium, platinum, ruthenium, iridium, tungsten, cobalt, vanadium, manganese, molybdenum, and other metals may be deposited by absorption, electroplating, magnetron and non-magnetron sputtering, electroless and other deposition techniques. The graphite solution is stirred for 10 seconds-48 h. The graphite solution is applied to surfaces by airbrush spray or hand brush or doctor blade or dip coating. The visual inspection of the coatings reveals that the coating appears smooth mat finish. The graphitic coating is largely homogeneous with thickness of 100 nanometers to 20 mm. The electrical sheet resistance of the devices was 0.1Ω-5000 Ωper square. Gram-positive (SIUC-11), Gram-negative (SIUC-2 and E. coli) bacteria, and Fusarium virguliforme with the concentration of 1 colony forming unit to 1×1010 cell forming units per mL were deactivated with the treatment of graphite devices with an applied potential 1 min to 24 h with an applied potential per centimeter square of the device of 0 V per cm2 to 10 V per cm2 (V per cm2 represent applied potential in volt divided by the area of the device). The distance between microbes and coatings can range from 0.1 nm to many meters. These coating particles are effective in deactivating microorganisms present on aerosols, water droplets, and gaseous/vapor conditions. This coating of microparticles may be applied to air and water filters, masks, textiles, filters for water decontamination, door and window knobs/handles, toilet seats and handles, equipment present in schools, university, hospital, industry, railway station, airport, agriculture, ships, airplanes, submarines, and homes. The coating of microparticles may also be applied to live plant pots to protect seeds and plants against bacteria, fungi, viruses, and archaea.

The device 10 may be any of various devices which may be broadly described as having a surface likely not to harbor undesirable microbes which may then be transferred to an individual who comes in contact with the surface, directly or indirectly. Such devices include clothing; bed linens, towels, masks intended to be worn by a human; ventilation systems and parts therefore, such as an air handler or an air filter.

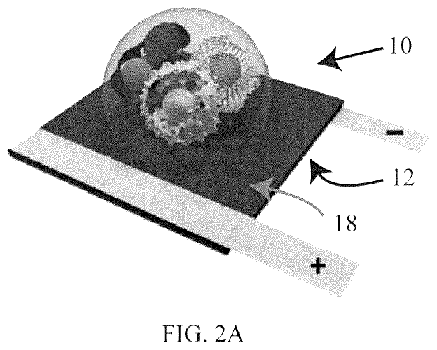

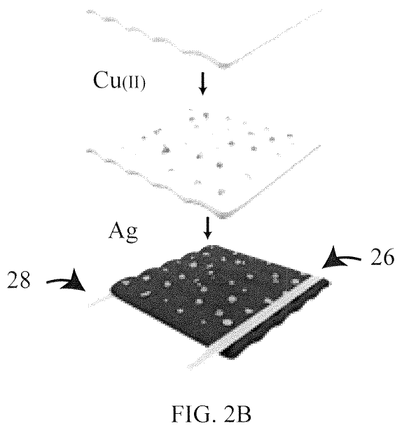

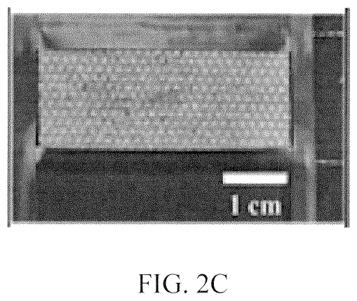

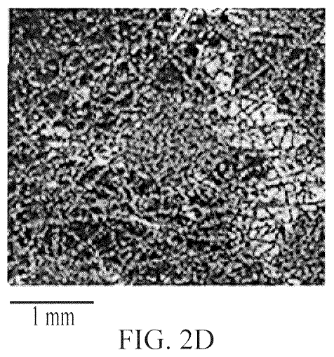

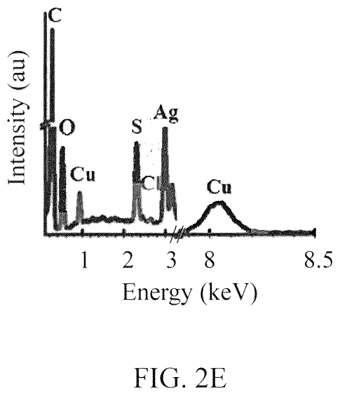

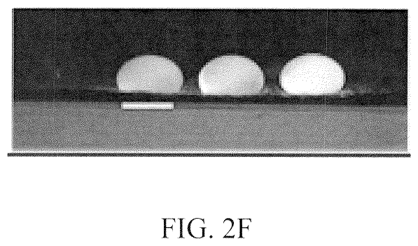

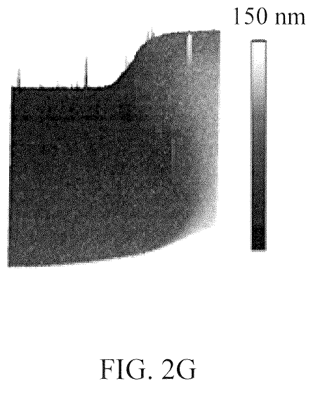

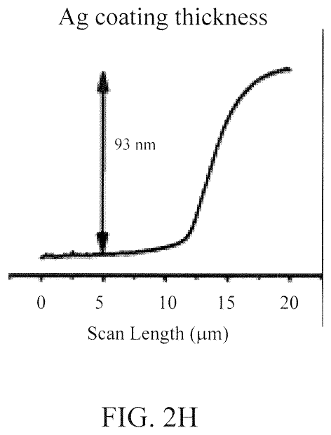

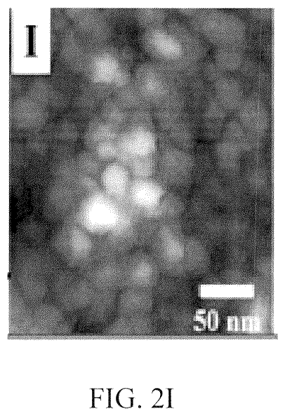

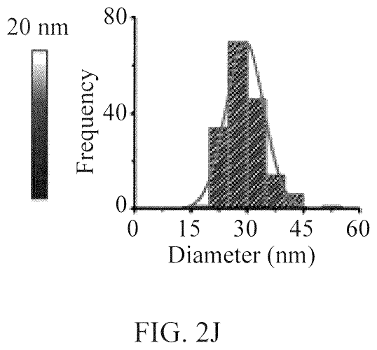

FIG. 2 illustrates an embodiment of the device body 12. The device body 12 in FIG. 2 provides a surface which may be incorporated in various devices for antimicrobial effect, including fabric-based article such as an article of clothing, a towel, and/or bed linens such as sheets and blankets, a filter mask, an item of medical equipment, a handheld electronic device, an item of processing equipment for a consumable, a ventilation system component, a wipe dispenser, a food preparation surface, an examination table for a human or an animal, a laboratory bench, a bathroom surface, a bathroom accessory, a personal care accessory and a hardware apparatus. The device 10 shown in FIG. 2 includes a plurality of antimicrobial metal components 18 Ag⁰ and Cu²⁺ disposed on the external surface of the raw mask material of a plurality of first electric element (in this example, a copper or silver lead), and a plurality of second elements (in this example, a Cu or silver lead). It is important to note that the plurality of antimicrobial metal components 18 are in a salt form. This is because the reactivity against microbes appear to come from Cu+, for example, which is formed reduction of Cu2+ through applied potential and the presence of Ag/AgCl.

In FIG. 2, each of the individual first and second elements are separated by a device body 12, which in this case is the raw mask material. The size of the device body 12 and thus the size of the separation between an individual first element and an individual second element is selected to optimize an antimicrobial effect.

A power source is connected to the device in FIG. 2B such that one terminal of the power source is in electrical communication with the first electrically conductive lead 26 and the other terminal of the power source is in electrical communication with the second electrically conductive lead 28.

### Example 1

FIG. 1 illustrates the device 10 according to the present invention. A first antimicrobial metal component 18 (Ag⁰) disposed in electrical connection with the device body 12 and a second antimicrobial metal component 18 (Cu²⁺) disposed in electrical connection with the device body 12 is shown. As shown in FIG. 1, the device body 12 is in electrical communication with a first terminal (+) of the power source through the first electrically conductive lead 26 and the device body 12 is in electrical communication with a second terminal (−) of the power source through the second electrically conductive lead 28. It is noted that the first electrically conductive lead 26 and second electrically conductive lead 28 are not in electrical contact except through the device body 12.

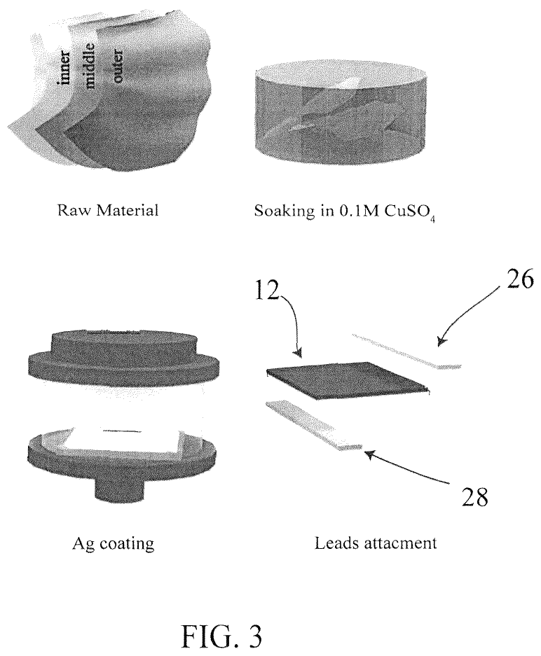

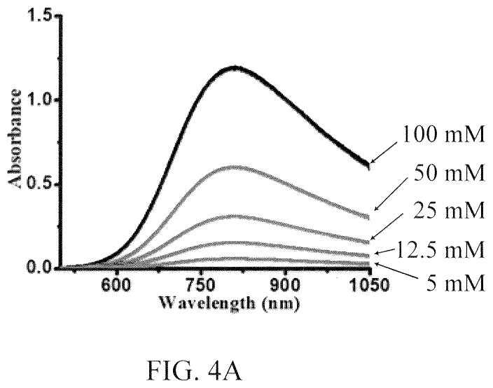

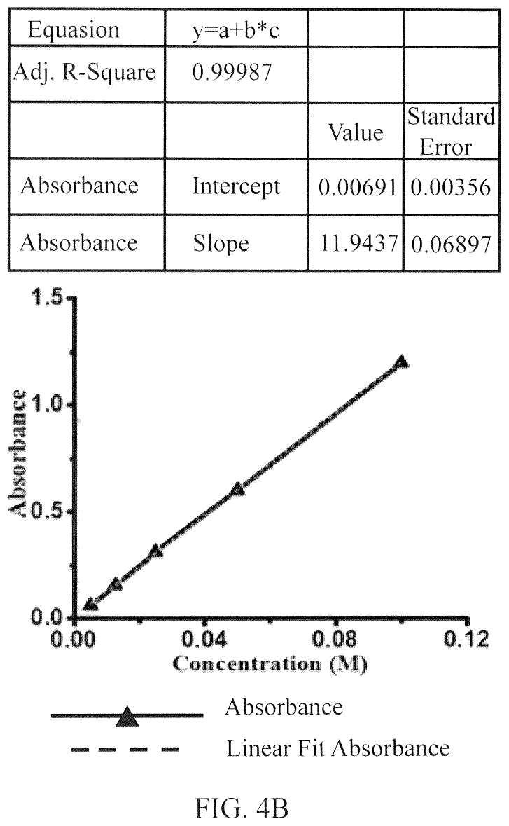

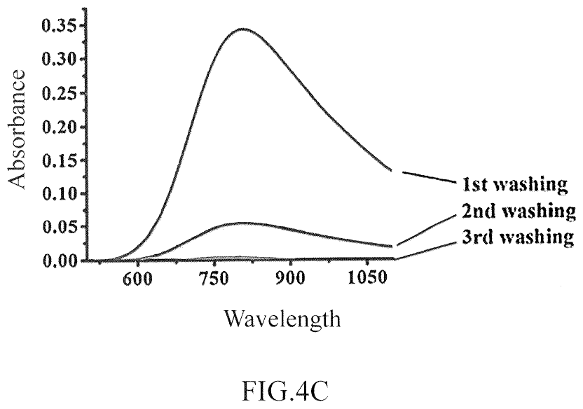

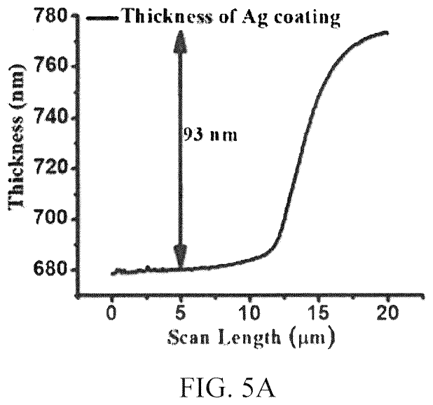

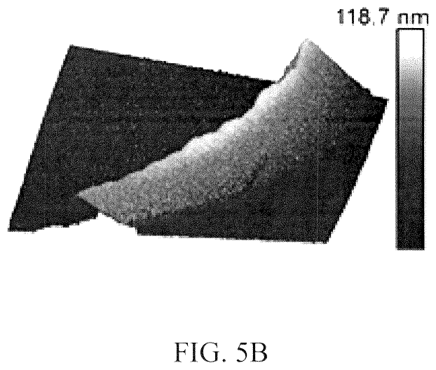

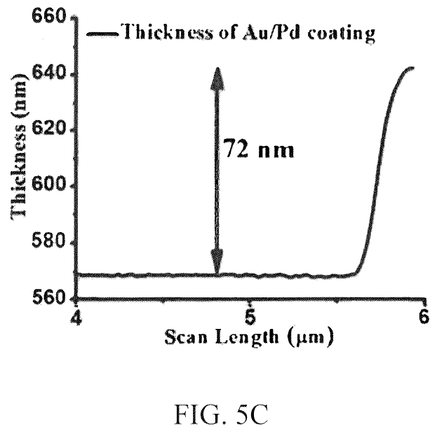

In a particular embodiment, the device body 12 was fabricated using the process shown in FIG. 2 and FIG. 3. First, the surface of the device body 12 was chosen. In the preferred embodiments described herein, such a surface may include a polypropylene fabric, house-hold air-filter, cellulose filter paper or hand paper. Next, the device body 12 was immersed in a 0.1 M CuSO₄ solution for one hour (FIG. 2B and FIG. 3). The Cu-containing surface was then dried under dry nitrogen and/or air yielding [Cu²⁺]≈15.7 ppm cm⁻² (FIGS. 3 and 4). Next, 80-100 nm thick silver was sputtered both sides of the Cu-containing device body 12 surface (FIG. 3) using a Denton Vacuum desk II. This yielded a nanoscale copper-silver coated device body 12 surface (FIG. 2G-I and FIG. 5). The device body 12 was then formed into the desired size. Conducting silver wires were then adhered to the device body 12 surface using silver paste. One wire was glued at the top side of the device 10 and another wire was glued at the bottom side of the device 10. The wires were then insulated using black electrical tape to ensure the device surfaces were electrically insulated from surrounding electrical interference. The electrical-polarization of the copper-silver coatings was achieved by attaching an external power source (1.5-9 V) to the device body 12 using silver wires. FIG. 2C shows an optical micrograph of a typical device body 12.

In other embodiments, Cu (Lₐ 0.93 keV and K_{α} 8.04 keV, respectively) and Ag (Lₐ 2.984 keV) was used in coating the device body 12 in the ratios set forth in Table 1.

TABLE 1
Elemental composition of modified surfaces by EDS

| Modified | | Energy | Average | Average |
|---|---|---|---|---|
| surfaces | Element | keV | Weight (Std) % | Atomic (Std) % |
| CuSO4 soaked | C | 0.27 | 86.23 (6.23) | 91.13 (4.01) |
| device body | O | 0.52 | 9.59 (3.98) | 7.72 (3.39) |
| surface | S | 2.30 | 1.49 (0.75) | 0.6 (0.31) |
| | Cu | 0.93 | 2.67 (1.52) | 0.54 (0.32) |
| Ag coated device | C | 0.27 | 73.62 (1.13) | 94.23 (0.23) |
| body surface | O | 0.52 | 2.45 (0.38) | 2.35 (0.34) |
| | Ag | 2.98 | 23.91 (1.41) | 3.41 (0.25) |
| Cu soaked and | C | 0.27 | 85.24 (1.67) | 93.07 (0.97) |
| Ag coated device | O | 0.52 | 6.54 (0.94) | 5.37 (0.82) |
| body surface | S | 2.30 | 0.98 (0.15) | 0.40 (0.06) |
| | Cu | 0.93; | 3.22 (0.48) | 0.66 (0.10) |
| | | 8.04 | | |
| | Ag | 2.98 | 3.99 (0.49) | 0.48 (0.06) |

### Example 2

In another example, the Cu-containing surface was sputter coated with silver for 6 minutes using a Denton Vacuum desk III and Au/Pd for 3 minutes using a Denton Vacuum desk II with a silver target respectively. The sputtering was performed for 6 minutes at a pressure of 50 mTorr and 40 mA. The thickness of silver surface is shown in FIG. 2G-I while the thickness of the gold and palladium surface is shown in FIG. 5, where the thin films were approximately 93 nm and 73 nm, respectively. These thin films provide an advantage over thicker films because these nanoscale surfaces are more effective against microbes. As such, it is advantageous to have the films thinner for a higher efficacy in deactivating microbes. Finally, the coated surface was cut into 4 cm×4 cm pieces. For each sample, conducting silver wires were adhered to the sample using silver paste. One wire was glued on the top side residing at the first external surface 14 of the device 10 and another wire was glued at the bottom side situated at the second external surface 16 of the device 10. The wires were then insulated using a black electrical tape to ensure the device surfaces were electrically insulated from surrounding electrical interference. The electrical-polarization of the copper-silver coatings is achieved by attaching the electrically conductive leads to an external power source using silver wires. Usually, an alkaline battery (1.5 V-9V) was employed as the external power source. In some cases, a solar cell was employed as the external power source such that the solar cell was irradiated with indoor lights. FIG. 2C shows an optical micrograph of a typical device. The device body 12 was fabricated using this method on a number of surfaces including mask fabric (polypropylene), house-hold air-filter, cellulose filter paper and hand paper.

Reactive oxygen species (ROS) play an important regulator role in cellular reproduction and cell death.^{38, 39} Some ROS are known to be highly toxic to microorganisms^{34, 35}. ROS are oxygen derived intermediates with higher reactivity and redox activity. ROS are classified as free radicals including superoxide radical (O₂^{•-}), hydroxyl radical (OH^{•}), hydroperoxyl radical (OH₂^{•}), and non-radicals such as hydrogen peroxide (H₂O₂) and singlet oxygen (¹O₂)³⁵. Superoxide radical (O₂^{•-}), hydroxyl radical (OH^{•}), singlet oxygen (¹O₂) and hydrogen peroxide (H₂O₂) produce high oxidative stress causing toxicity, deactivation, and killing in microorganisms^{29, 34, 35}. Moreover, the cell cycle can also be affected by the ROS concentration. For example, microorganism exposed to a moderate concentration of ROS may exhibit cell cycle inhibition, whereas high concentration of ROS may result in cell death³⁶. ROS based antimicrobial properties and mechanism of action are still an active interest of research area. This work demonstrates that the broad-band high deactivation effectiveness of electrically-polarized metallic devices is dominant by generation of micromolar ROS (H₂O₂, OH^{•}, and O₂^{•-}) through redox Cu(0/I/II) coupling with O₂ and H₂O. The generation of Cu(I) is key to the production of high concentration of H₂O₂ in the solution through oxygen reduction reaction mediated by cuprous ions. The spectroscopic production of nanomolar OH^{•} in the solution was also observed. OH^{•} is known to be highly damaging and detrimental to cells and will kill cells immediately on contact.

The device body 12 surface works to produce Cu(I) through two different mechanisms, First, a Walden reductor (Ag+Cl⁻ →AgCl(s)+e⁻) is employed to generate Cu(I) by reducing Cu(II) to Cu(I).^{34, 35} This reaction generates Cu(I) in the electrical nonpolarized metallic device body 12 (Eₐₚₚ=0V). The dominant mechanism of generating Cu(I) is done through electrically-polarizing the Cu²⁺—Ag through applied electrical potential (Eₐₚₚ=1.5V-6V). Cu(I) is produced at the cathode through one-electron reduction process in CuSO₄ and CuCl₂ solutions, whereas a two-electron reduction process yields Cu(0).⁴¹

H₂O₂ was produced by two-step pathways, where the first step involves oxygen reduction to superoxide (Equations 1 and 2 below), followed by H₂O₂ production either by superoxide disproportional (Equation 4) or reaction with H⁺ mediated through Cu(I) (Equation 5).

$${Cu}^{+} + {O}_{2} \to Cu ( II ) + {O}_{2}^{\cdot –} {k}_{1} = 3.1 \times {10}^{4} {M}^{- 1} {s}^{- 1} \qquad Equation 1$$

$${CuClOH}^{–} + {O}_{2} \to Cu ( II ) + {O}_{2}^{\cdot –} {k}_{2} = 3.1 \times {10}^{2} {M}^{- 1} {s}^{- 1} \qquad Equation 2$$

$${CuCO}_{3}^{-} + {O}_{2} \to Cu ( II ) + {O}_{2}^{\cdot –} {k}_{3} = 1. \times 1 {0}^{4} {M}^{- 1} {s}^{- 1} \qquad Equation 3$$

The existence of intermediate such as O₂^{•-} and H₂O₂ is confirmed through the following equations:

$${O}_{2}^{\cdot –} + {O}_{2}^{\cdot –} + 2 {H}^{+} \to {H}_{2} {O}_{2} + {O}_{2} {k}_{4} = 8.3 \times {10}^{5} {M}^{- 1} {s}^{- 1} ( pH = 7 ) \qquad Equation 4$$

$$Cu ( I ) + {O}_{2}^{\cdot –} + 2 {H}^{+} \to Cu ( II ) + {H}_{2} {O}_{2} {k}_{5} 2 \times {10}^{9} {M}^{- 1} {s}^{- 1} \qquad Equation 5$$

$$Cu ( II ) + {O}_{2}^{\cdot –} \to Cu ( I ) + {O}_{2} {k}_{6} = 6.6 \times {10}^{9} {M}^{- 1} {s}^{- 1} \qquad Equation 6$$

$${Ag}^{+} + {O}_{2}^{\cdot –} \to AgNP + {O}_{2} {k}_{8} = 64.5 ( \pm 16.3 ) \qquad Equation 8$$

$$AgNP + {O}_{2}^{\cdot –} \to {AgNP}^{*} + {O}_{2} {k}_{9} = 1 \times {10}^{10} {M}^{- 1} {s}^{- 1} \qquad Equation 9$$

$$2 Cu ( I ) + {O}_{2} + 2 H + \to 2 Cu ( II ) + {H}_{2} {O}_{2} {k}_{5}^{’} = 6.5 \times 1 {0}^{3} {M}^{- 1} {s}^{- 1} , \qquad Equation 9$$

The potential reactions involving consumption of H₂O₂ and generation of ^{•}OH:

$$Cu ( I ) + {H}_{2} {O}_{2} \to Cu ( I I ) + \cdot OH + {OH}^{-} {k}_{10} = 1. \times {10}^{2} {M}^{- 1} {s}^{- 1} \qquad Equation 10$$

$$Cu ( II ) + {H}_{2} {O}_{2} \to Cu ( I ) + {O}_{2}^{\cdot –} {k}_{11} < 1 {M}^{- 1} {s}^{- 1} \qquad Equation 11$$

$$AgNP + {{H}_{2} O}_{2} \to products {k}_{12} = 2.7 \times {10}^{2} {M}^{- 1} {s}^{- 1} ( size dependent ) \qquad Equation 12$$

$${O}_{2}^{\cdot –} + {H}_{2} {O}_{2} \to {O}_{2} + \cdot OH + {OH}^{–} {k}_{13} negligible for practical purposes \qquad Equation 13$$

$${H}_{2} {O}_{2} + {Cl}^{–} + Cu ( II ) \to HOCl + Cu ( I ) + {H}_{2} O {k}_{14} ( value was not given ) , \qquad Equation 13$$

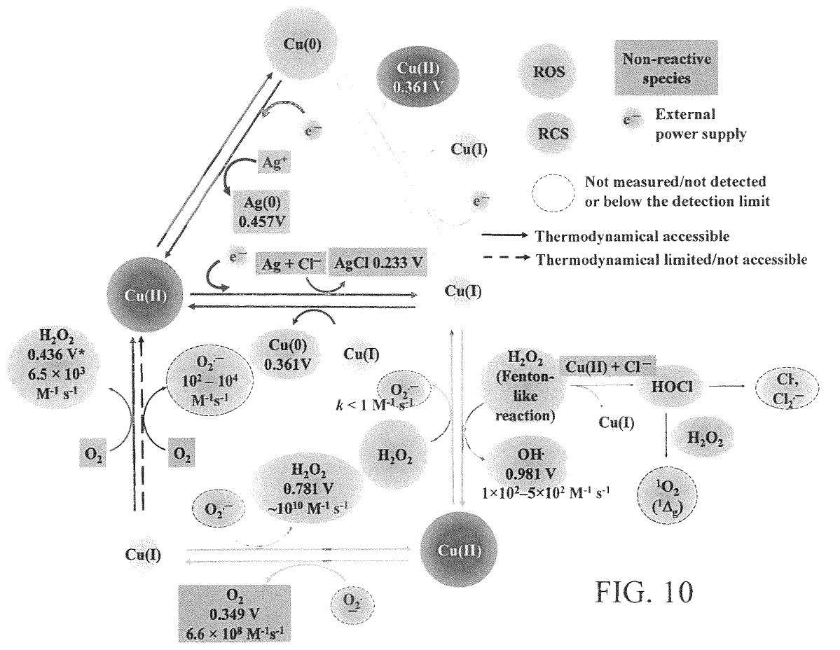

Equation 5 appears to be the dominant pathway for the production of H₂O₂ in this study. Interestingly, this mechanism requires two Cu(I) species within the diffusion distance during half-life of O₂^{•-}. In any case, as demonstrated above, H₂O₂ produced in micromolar concentration through electrical-polarization of the nanoscale metallic was sufficiently high enough for the deactivation of a broadband microbes with high efficiency within <10 min. The detection of [O₂^{•-}] through the reduction of ferri-cytochrome c to ferro-cytochrome c was not successful. The rationale behind this finding is because of extremely low [O₂^{•-}](in tens to hundred picomolar concentration) in the solution. The estimated [O₂^{•-}] in picomolar which is many orders of magnitude lower than [Cu(I)] and [H₂O₂] under similar experimental conditions. The significant lower [O₂^{•-}] is likely due to multiple deactivation pathways available for O₂^{•-} where it can react with other species including Cu(I), Cu(II), Cl⁻, O₂^{•-} and H₂O₂ etc. (FIG. 10). Interestingly, Equation 9′ represents a two-electron O₂ reduction reaction, which can also produce H₂O₂. This reaction is thermodynamically accessible (E₀=0.436 V), but it possesses a low-rate constant (k₅′=6.5×10³ M⁻¹ s⁻¹). Further, two closely located Cu(I) species are necessary for a single two-electron O₂ reduction to H₂O₂. With steady-state [Cu⁺]≈30 μM cm⁻², the probability of availability of two Cu⁺ in close proximity in bulk solution for the two-electron reduction reaction is expected to be small, although this reaction can be feasible when Cu²⁺ is aggregated on the solid surfaces. In any case, Cu⁺ plays a crucial role in the generation of H₂O₂ in both the mechanisms. However, due to a lack of clarity, we consider both the reaction mechanisms can be operable in the production of H₂O₂ in the ENM treatment (FIG. 10).

Cu(I) mediated reduction of oxygen generates O₂^{•-}, which produces H₂O₂ through O₂^{•-} disproportion reaction (Equation 4) or reaction with water mediated by Cu(I) (Equation 5). Waite described in detail oxygen reduction by Cu(I) occurs through three major reaction pathways at circumneutral pH (Equations 1-3, purple arrowed reaction in FIG. 10).³⁷ kᵢ are second order rate constants, which were taken from literature.³⁷⁻³⁹

Whereas the first two reactions are likely available in the present studies, the third reaction is likely to contribute negligible to the production of O₂^{•-} because of the absence of bicarbonate in our experiments. Equations 1 and 2 are used to describe O₂ reduction with Cu(I). In general, the reduction of O₂ with copper species is usually expressed in terms of total Cu(I) and Cu(II) concentrations, rather than explicit individual copper species (Cu⁺ and Cu²⁺). This is because of Cu(I) stabilization due to complex formation with ligands (such as Cl⁻) in the solution.^{37, 38}

Knowing that k₂«k₁ and that the contribution of Equation 3 to O₂^{•-} production is small, the transient O₂^{•-} concentration (denoted by [O₂^{•-}]ₜᵣ) is ~k₁[O₂^{•-}][Cu(I)]≈k₁′[Cu(I)]. Here, “[O₂]” and “kᵢ′” represent the O₂ concentration in the air saturated water (0.28 mM) and pseudo-first order rate constant, respectively. O₂^{•-} reacts with Cu(I) and Cu(II) with diffusion-limited rate constants of 2×10⁹ M⁻¹s⁻¹ (Equation 5) and 6.6×10⁸ M⁻¹s⁻¹ (Equation 6), respectively. These reactions will dissipate O₂^{•-} rapidly decreasing [O₂^{•-}]ₜᵣ in the solution. It is determined that the steady state O₂^{•-} concentration (denoted by [O₂^{•-}]ₛₛ) is in the 14 μM-234 μM range, the same as in sunlit seawaters.⁴⁰ Extremely low [O₂^{•-}]ₛₛ in picomolar range is consistent with the experimental results, where the detection of O₂^{•-} was not conclusive in the experiments using reduction of cytochrome C by O₂^{•-}. Silver nanoparticles (AgNPs) can also react with O₂^{•-} with diffusion-limited rate constants (k=1×10¹⁰ M⁻¹s⁻¹), providing another potential draining pathway for [O₂^{•-}] in these experiments.⁴¹ The contribution of Ag⁺ (formed through oxidation of Ag at the positive electrode) to O₂^{•-} consumption (Equation 8) is negligible because of extremely slow reaction kinetics (k₈=64.5 M⁻¹s⁻¹).⁴¹ The reaction of O₂^{•-} with Cu(I) and Cu(II) are fast, implying that that the O₂^{•-} consumption is dominated by Equations 5 and 6, and that superoxide disproportion reaction (Equation 4) contributes insignificantly to its consumption. Importantly, H₂O₂ was generated by O₂^{•-} disproportion (Equation 4) and O₂^{•-}/H⁺ reaction (Equation 5). At long duration of time (τₐₚₚ≈24 h), AgNP may also play role in the formation and dissociation of H₂O₂.⁴¹ The reaction between O₂^{•-} and H₂O mediated with Cu(I) (Equation 5), and the disproportion O₂^{•-} reaction (Equation 4) are two major routes for the generation of H₂O₂ with rate constants 2×10⁹ M⁻¹s⁻¹ and 7.0×10⁵ M⁻¹s⁻¹ respectively. With k₄ is more than three orders of magnitude lower than k₅, it implies that H₂O₂ production is dominant by Equation 5 and that the O₂^{•-} disproportion reaction is believed to be a minor H₂O₂ contributor. The results indicate that the concentration of Cu(I) is a crucial parameter for the generation of O₂^{•-} and H₂O₂. Overall, the interplay of Equations 1, 2, 5 and 6 appear to dictate the steady state [H₂O₂] in the solution in the initial stage of reaction.

FIG. 10 has two major parts showing redox pathways for the generation of ROS (and RCS) and Cu(0/I/II) cycling in the device body 12. The bottom square emphasizes the production of ROS (and RCS) mediated by the copper redox chemistry. The redox pathways in the top triangular portion are intended to emphasize copper redox chemistry, where Cu(0), Cu⁺, and Cu²⁺ form three vertices of a triangle. Cu⁺ and Cu²⁺, but not Cu(I) and Cu(II), are used in the figure because half-cell reduction potentials of the former copper species are well-known in the literature, although similar redox pathways are available for Cu(I) and Cu(II) species. Here, Cu(I) and Cu(II) represent the sum of all cuprous and cupric species, respectively in the solution.

$${Cu}^{2 +} + {e}^{-} \to {Cu}^{+} {E}_{0} = 0.159 V \qquad Equation 14$$

$$Cu \to {Cu}^{+} + {e}^{–} {E}_{0} = - 0.52 V \qquad Equation 15$$

$${Cu}^{2 +} + Ag + 3 {Cl}^{–} \to {CuCl}_{2}^{–} + AgCl {E}_{0} = 0.223 V \qquad Equation 16$$

$${Cu}^{+} + {Cu}^{+} \to {Cu}^{2 +} + Cu {E}_{0} = 0.361 V \qquad Equation 17$$

$${Cu}^{2 +} + 2 {e}^{–} \to Cu {E}_{0} = 0.3419 V \qquad Equation 18$$

$$Ag \to {Ag}^{+} + {e}^{–} {E}_{0} = - 0.7996 V \qquad Equation 19$$

$${Ag}^{+} + Cu \to Ag + {Cu}^{2 +} {E}_{0} = 0.4575 \qquad Equation 20$$

) As discussed earlier, there are two major Cu(I) sources in the device body 12—reduction of Cu²⁺ at the negative electrode (Equation 14), and (2) reduction of Cu²⁺ to Cu⁺ through Walden reductor (Equation 16). However, the degree of Cu⁺ generated in the device body 12 is much higher when realized through the application of electrical potential (Eₐₚₚ=1.5V−6V, Equation 15) using an external battery. Cu⁺ is known to form at the cathode through an one-electron reduction process of Cu²⁺ (Equation 14), whereas a two electron reduction process yields Cu(0) (Equation 18).⁴² It is expected that these two reactions would occur simultaneously at the negative electrode of the device body 12. The Cu⁺ production for Ag-coated devices also exhibited a similar trend—showing a much larger steady state [Cu(I)]≈6 μM (τₐₚₚ=2 h) for Eₐₚₚ=3 V but yielded a negligible [Cu⁺] for Eₐₚₚ=0 V (τₐₚₚ=2 h). Collectively, these results indicated that electrical polarization is the dominant mechanism for the generation of Cu⁺ in the system, although Walden reaction also contributed to the production of Cu⁺ in the solution.

The Cu⁺ consumption is expected to occur through following pathways: First, the disproportion reaction (E₀=0.361 V, Equation 17) is a thermodynamically favorable reaction converting Cu⁺ into Cu(0) and Cu²⁺. However, Cu⁺—Cl complexes are more stable to copper disproportion reaction, which forms stable complexes such as CuCl(s), CuCl₂⁻, and CuCl₃²⁻ in the solution with formation constants of 1.3×10³, 4.8×10⁵, and 1.1×10⁵ respectively.³⁷ A second route that involves Cu⁺ dissipation is through many redox pathways for the production of ROS as described above (square part of FIG. 10). Interestingly, Cu(I) is involved in the production of O₂^{•-}, H₂O₂ and OH^{•} (FIG. 10B). Finally, the oxidation of Ag(0) yielded Ag⁺ at the positive electrode (Equation 19), which reacts spontaneously with Cu(0) (Equation 20, E₀=0.4575V). This reaction is important in converting Cu(0) to Cu²⁺. The Cu(0) to Cu²⁺ reaction also occurs at the positive device electrode. The later reaction and Ag⁺/Cu(0) reaction reduce Cu(0) precipitation in the solution. Although, more details studies are needed to fully evaluate the Cu(0)/Cu(I)/Cu(II) cycling in the ROS devices, the interplay of multiple redox reactions yielded micromolar Cu(I) concentration in the solution which was sufficiently high to producing micromolar H₂O₂ for the deactivation of broad-band microbes.

While several particular embodiments of the present invention have been described herein, it will be appreciated by those skilled in the art that changes and modifications may be made thereto without departing from the invention in its broader aspects and as set forth in the following claims.

### REFERENCES

(1) Egger, D.; Miguel, E.; Warren, S. S.; Shenoy, A.; Collins, E.; Karlan, D.; Parkerson, D.; Mobarak, A. M.; Fink, G.; Udry, C.; et al. Falling living standards during the COVID-19 crisis: Quantitative evidence from nine developing countries. Science Advances 2021, 7 (6), eabe0997. DOI: doi:10.1126/sciadv.abe0997. Balasubramaniam, B.; Prateek; Ranjan, S.; Saraf, M.; Kar, P.; Singh, S. P.; Thakur, V. K.; Singh, A.; Gupta, R. K. Antibacterial and Antiviral Functional Materials: Chemistry and Biological Activity toward Tackling COVID-19-like Pandemics. ACS Pharmacology & Translational Science 2021, 4 (1), 8-54. DOI: 10.1021/acsptsci.0c00174. Wu, D.; Wu, T.; Liu, Q.; Yang, Z. The SARS-CoV-2 outbreak: What we know. International Journal of Infectious Diseases 2020, 94, 44-48. DOI: https://doi.org/10.1016/j.ijid.2020.03.004.
(2) Bunge, E. M.; Hoet, B.; Chen, L.; Lienert, F.; Weidenthaler, H.; Baer, L. R.; Steffen, R. The changing epidemiology of human monkeypox-A potential threat? A systematic review. PLoS neglected tropical diseases 2022, 16 (2), e0010141. Thornhill, J. P.; Barkati, S.; Walmsley, S.; Rockstroh, J.; Antinori, A.; Harrison, L. B.; Palich, R.; Nori, A.; Reeves, I.; Habibi, M. S.; et al. Monkeypox Virus Infection in Humans across 16 Countries—April-June 2022. New England Journal of Medicine 2022, 387 (8), 679-691. DOI: 10.1056/NEJMoa2207323.
(3) Viboud, C.; Bjørnstad, O. N.; Smith, D. L.; Simonsen, L.; Miller, M. A.; Grenfell, B. T. Synchrony, Waves, and Spatial Hierarchies in the Spread of Influenza. Science 2006, 312 (5772), 447-451. DOI: doi:10.1126/science.1125237. Cowling, B. J.; Ip, D. K. M.; Fang, V. J.; Suntarattiwong, P.; Olsen, S. J.; Levy, J.; Uyeki, T. M.; Leung, G. M.; Malik Peiris, J. S.; Chotpitayasunondh, T.; et al. Aerosol transmission is an important mode of influenza A virus spread. Nature Communications 2013, 4 (1), 1935. DOI: 10.1038/ncomms2922. Du, X.; King, A. A.; Woods, R. J.; Pascual, M. Evolution-informed forecasting of seasonal influenza A (H3N2). Science Translational Medicine 2017, 9 (413), eaan5325. DOI: doi:10.1126/scitranslmed.aan5325.
(4) Düx, A.; Lequime, S.; Patrono, L. V.; Vrancken, B.; Boral, S.; Gogarten, J. F.; Hilbig, A.; Horst, D.; Merkel, K.; Prepoint, B.; et al. Measles virus and rinderpest virus divergence dated to the sixth century BCE. Science 2020, 368 (6497), 1367-1370. DOI: doi:10.1126/science.aba9411. Yang, W. Transmission dynamics of and insights from the 2018& #x2013; 2019 measles outbreak in New York City: A modeling study. Science Advances 2020, 6 (22), eaaz4037. DOI: doi:10.1126/sciadv.aaz4037.
(5) Michael, C. A.; Dominey-Howes, D.; Labbate, M. The Antimicrobial Resistance Crisis: Causes, Consequences, and Management. Frontiers in Public Health 2014, 2, Review. DOI: 10.3389/fpubh.2014.00145.
(6) Organization, W. H. Antimicrobial resistance: global report on surveillance; World Health Organization, 2014. Micoli, F.; Bagnoli, F.; Rappuoli, R.; Serruto, D. The role of vaccines in combatting antimicrobial resistance. Nature Reviews Microbiology 2021, 19 (5), 287-302. DOI: 10.1038/s41579-020-00506-3. Lipsitch, M.; Samore, M. H. Antimicrobial use and antimicrobial resistance: a population perspective. Emerging infectious diseases 2002, 8 (4), 347.
(7) Cloutier, M.; Mantovani, D.; Rosei, F. Antibacterial Coatings: Challenges, Perspectives, and Opportunities. Trends in Biotechnology 2015, 33 (11), 637-652. DOI: https://doi.org/10.1016/i.tibtech.2015.09.002.
(8) Salwiczek, M.; Qu, Y.; Gardiner, J.; Strugnell, R. A.; Lithgow, T.; McLean, K. M.; Thissen, H. Emerging rules for effective antimicrobial coatings. Trends in Biotechnology 2014, 32 (2), 82-90. DOI: https://doi.org/10.1016/i.tibtech.2013.09.008.
(9) Knetsch, M. L.; Koole, L. H. New strategies in the development of antimicrobial coatings: the example of increasing usage of silver and silver nanoparticles. Polymers 2011, 3 (1), 340-366.
(10) Tiller, J. C. Antimicrobial surfaces. Bioactive surfaces 2011, 193-217.
(11) Hamley, I. W. Small bioactive peptides for biomaterials design and therapeutics. Chemical reviews 2017, 117 (24), 14015-14041. Eckhardt, S.; Brunetto, P. S.; Gagnon, J.; Priebe, M.; Giese, B.; Fromm, K. M. Nanobio silver: its interactions with peptides and bacteria, and its uses in medicine. Chemical reviews 2013, 113 (7), 4708-4754.
(12) Song, B.; Zhang, E.; Han, X.; Zhu, H.; Shi, Y.; Cao, Z. Engineering and Application Perspectives on Designing an Antimicrobial Surface. ACS Applied Materials & Interfaces 2020, 12 (19), 21330-21341. DOI: 10.1021/acsami.9b19992.
(13) Garland, M.; Loscher, S.; Bogyo, M. Chemical strategies to target bacterial virulence. Chemical reviews 2017, 117 (5), 4422-4461.
(14) Ren, G.; Hu, D.; Cheng, E. W.; Vargas-Reus, M. A.; Reip, P.; Allaker, R. P. Characterisation of copper oxide nanoparticles for antimicrobial applications. International journal of antimicrobial agents 2009, 33 (6), 587-590.
(15) Krishnamoorthy, M.; Hakobyan, S.; Ramstedt, M.; Gautrot, J. E. Surface-Initiated Polymer Brushes in the Biomedical Field: Applications in Membrane Science, Biosensing, Cell Culture, Regenerative Medicine and Antibacterial Coatings. Chemical Reviews 2014, 114 (21), 10976-11026. DOI: 10.1021/cr500252u. Kenawy, E. R.; Worley, S.; Broughton, R. The chemistry and applications of antimicrobial polymers: a state-of-the-art review. Biomacromolecules 2007, 8 (5), 1359-1384.
(16) Kim, J. S.; Kuk, E.; Yu, K. N.; Kim, J. H.; Park, S. J.; Lee, H. J.; Kim, S. H.; Park, Y. K.; Park, Y. H.; Hwang, C. Y.; et al. Antimicrobial effects of silver nanoparticles. Nanomedicine: Nanotechnology, Biology and Medicine 2007, 3 (1), 95-101. DOI: https://doi.org/10.1016/j.nano.2006.12.001.
(17) Li, Y.; Zhang, W.; Niu, J.; Chen, Y. Mechanism of photogenerated reactive oxygen species and correlation with the antibacterial properties of engineered metal-oxide nanoparticles. ACS nano 2012, 6 (6), 5164-5173.
(18) Ruparelia, J. P.; Chatterjee, A. K.; Duttagupta, S. P.; Mukherji, S. Strain specificity in antimicrobial activity of silver and copper nanoparticles. Acta biomaterialia 2008, 4 (3), 707-716.
(19) Rai, M.; Yadav, A.; Gade, A. Silver nanoparticles as a new generation of antimicrobials. Biotechnology Advances 2009, 27 (1), 76-83. DOI: https://doi.org/10.1016/i.biotechadv.2008.09.002.
(20) Leung, Y. H.; Ng, A. M. C.; Xu, X.; Shen, Z.; Gethings, L. A.; Wong, M. T.; Chan, C. M. N.; Guo, M. Y.; Ng, Y. H.; Djurišić, A. B.; et al. Mechanisms of Antibacterial Activity of MgO: Non-ROS Mediated Toxicity of MgO Nanoparticles Towards Escherichia coli. Small 2014, 10 (6), 1171-1183. DOI: https://doi.org/10.1002/smll.201302434.
(21) Arakha, M.; Pal, S.; Samantarrai, D.; Panigrahi, T. K.; Mallick, B. C.; Pramanik, K.; Mallick, B.; Jha, S. Antimicrobial activity of iron oxide nanoparticle upon modulation of nanoparticle-bacteria interface. Scientific Reports 2015, 5 (1), 14813. DOI: 10.1038/srep14813.
(22) Jesline, A.; John, N. P.; Narayanan, P.; Vani, C.; Murugan, S. Antimicrobial activity of zinc and titanium dioxide nanoparticles against biofilm-producing methicillin-resistant Staphylococcus aureus. Applied Nanoscience 2015, 5, 157-162. Long, T. C.; Saleh, N.; Tilton, R. D.; Lowry, G. V.; Veronesi, B. Titanium Dioxide (P25) Produces Reactive Oxygen Species in Immortalized Brain Microglia (BV2): Implications for Nanoparticle Neurotoxicity. Environmental Science & Technology 2006, 40 (14), 4346-4352. DOI: 10.1021/es060589n.
(23) Heinlaan, M.; Ivask, A.; Blinova, I.; Dubourguier, H. C.; Kahru, A. Toxicity of nanosized and bulk ZnO, CuO and TiO2 to bacteria Vibrio fischeri and crustaceans Daphnia magna and Thamnocephalus platyurus. Chemosphere 2008, 71 (7), 1308-1316. DOI: https://doi.org/10.1016/i.chemosphere.2007.11.047. Baek, Y. W.; An, Y. J. Microbial toxicity of metal oxide nanoparticles (CuO, NiO, ZnO, and Sb2O3) to Escherichia coli, Bacillus subtilis, and Streptococcus aureus. Science of The Total Environment 2011, 409 (8), 1603-1608. DOI: https://doi.org/10.1016/i.scitotenv.2011.01.014.
(24) Xia, T.; Kovochich, M.; Liong, M.; Madler, L.; Gilbert, B.; Shi, H.; Yeh, J. I.; Zink, J. I.; Nel, A. E. Comparison of the Mechanism of Toxicity of Zinc Oxide and Cerium Oxide Nanoparticles Based on Dissolution and Oxidative Stress Properties. ACS Nano 2008, 2 (10), 2121-2134. DOI: 10.1021/nn800511k.
(25) Emami-Karvani, Z.; Chehrazi, P. Antibacterial activity of ZnO nanoparticle on gram-positive and gram-negative bacteria. Afr J Microbiol Res 2011, 5 (12), 1368-1373.
(26) Hajipour, M. J.; Fromm, K. M.; Akbar Ashkarran, A.; Jimenez de Aberasturi, D.; Larramendi, I. R. d.; Rojo, T.; Serpooshan, V.; Parak, W. J.; Mahmoudi, M. Antibacterial properties of nanoparticles. Trends in Biotechnology 2012, 30 (10), 499-511. DOI: https://doi.org/10.1016/j.tibtech.2012.06.004. Wang, L.; Hu, C.; Shao, L. The antimicrobial activity of nanoparticles: present situation and prospects for the future. International journal of nanomedicine 2017, 12, 1227.
(27) Dizaj, S. M.; Lotfipour, F.; Barzegar-Jalali, M.; Zarrintan, M. H.; Adibkia, K. Antimicrobial activity of the metals and metal oxide nanoparticles. Materials Science and Engineering: C 2014, 44, 278-284. DOI: https://doi.org/10.1016/i.msec.2014.08.031.
(28) Nosaka, Y.; Nosaka, A. Y. Generation and Detection of Reactive Oxygen Species in Photocatalysis. Chemical Reviews 2017, 117 (17), 11302-11336. DOI: 10.1021/acs.chemrev.7b00161.
(29) Winterbourn, C. C. Reconciling the chemistry and biology of reactive oxygen species. Nature Chemical Biology 2008, 4 (5), 278-286. DOI: 10.1038/nchembio.85.
(30) Hancock, J.; Desikan, R.; Neill, S. Role of reactive oxygen species in cell signalling pathways. Biochemical Society Transactions 2001, 29 (2), 345-349. Murphy, M. P. How mitochondria produce reactive oxygen species. Biochemical journal 2009, 417 (1), 1-13. Thannickal, V. J.; Fanburg, B. L. Reactive oxygen species in cell signaling. American Journal of Physiology-Lung Cellular and Molecular Physiology 2000, 279 (6), L1005-L1028.
(31) Turrens, J. F. Mitochondrial formation of reactive oxygen species. The Journal of Physiology 2003, 552 (2), 335-344. DOI: https://doi.org/10.1111/j.1469-7793.2003.00335.x. Finkel, T. Signal transduction by reactive oxygen species. Journal of Cell Biology 2011, 194 (1), 7-15. DOI: 10.1083/jcb.201102095 (acccessed Mar. 15, 2023).
(32) Si, Y.; Zhang, Z.; Wu, W.; Fu, Q.; Huang, K.; Nitin, N.; Ding, B.; Sun, G. Daylight-driven rechargeable antibacterial and antiviral nanofibrous membranes for bioprotective applications. Science Advances 2018, 4 (3), eaar5931. DOI: doi:10.1126/sciadv.aar5931.
(33) Santos, A. L.; Liu, D.; Reed, A. K.; Wyderka, A. M.; van Venrooy, A.; Li, J. T.; Li, V. D.; Misiura, M.; Samoylova, O.; Beckham, J. L.; et al. Light-activated molecular machines are fast-acting broad-spectrum antibacterials that target the membrane. Science Advances 2022, 8 (22), eabm2055. DOI: doi:10.1126/sciadv.abm2055.
(34) Tang, S.; Zheng, J. Antibacterial Activity of Silver Nanoparticles: Structural Effects. Advanced Healthcare Materials 2018, 7 (13), 1701503. DOI: https://doi.org/10.1002/adhm.201701503. Deshmukh, S. P.; Patil, S. M.; Mullani, S. B.; Delekar, S. D. Silver nanoparticles as an effective disinfectant: A review. Materials Science and Engineering: C 2019, 97, 954-965. DOI: https://doi.org/10.1016/i.msec.2018.12.102. Sondi, I.; Salopek-Sondi, B. Silver nanoparticles as antimicrobial agent: a case study on E. coli as a model for Gram-negative bacteria. Journal of Colloid and Interface Science 2004, 275 (1), 177-182. DOI: https://doi.org/10.1016/j.jcis.2004.02.012. Chatterjee, A. K.; Chakraborty, R.; Basu, T. Mechanism of antibacterial activity of copper nanoparticles. Nanotechnology 2014, 25 (13), 135101. DOI: 10.1088/0957-4484/25/13/135101. Santo, C. E.; Quaranta, D.; Grass, G. Antimicrobial metallic copper surfaces kill Staphylococcus haemolyticus via membrane damage. Microbiology open 2012, 1 (1), 46-52. Meghana, S.; Kabra, P.; Chakraborty, S.; Padmavathy, N. Understanding the pathway of antibacterial activity of copper oxide nanoparticles. RSC Advances 2015, 5 (16), 12293-12299, 10.1039/C4RA12163E. DOI: 10.1039/c4ra12163e. Yin, I. X.; Zhang, J.; Zhao, I. S.; Mei, M. L.; Li, Q.; Chu, C. H. The Antibacterial Mechanism of Silver Nanoparticles and Its Application in Dentistry. International journal of nanomedicine 2020, 15, 2555-2562. DOI: 10.2147/ijn.s246764 PubMed. Xu, Z.; Zhang, C.; Wang, X.; Liu, D. Release Strategies of Silver Ions from Materials for Bacterial Killing. ACS Applied Bio Materials 2021, 4 (5), 3985-3999. DOI: 10.1021/acsabm.0c01485.
(35) Fang, F. C. Antimicrobial reactive oxygen and nitrogen species: concepts and controversies. Nature Reviews Microbiology 2004, 2 (10), 820-832. DOI: 10.1038/nrmicro1004. Gomes, A.;
Fernandes, E.; Lima, J. L. F. C. Fluorescence probes used for detection of reactive oxygen species. Journal of Biochemical and Biophysical Methods 2005, 65 (2), 45-80. DOI: https://doi.org/10.1016/j.jbbm.2005.10.003.
(36) Zhang, Y.; Dai, M.; Yuan, Z. Methods for the detection of reactive oxygen species. Analytical Methods 2018, 10 (38), 4625-4638, 10.1039/C8AY01339J. DOI: 10.1039/c8ay01339j. Hong, Y.; Zeng, J.; Wang, X.; Drlica, K.; Zhao, X. Post-stress bacterial cell death mediated by reactive oxygen species. Proceedings of the National Academy of Sciences 2019, 116 (20), 10064. DOI: 10.1073/pnas.1901730116. Zhao, X.; Drlica, K. Reactive oxygen species and the bacterial response to lethal stress. Current Opinion in Microbiology 2014, 21, 1-6. DOI: https://doi.org/10.1016/j.mib.2014.06.008.
(37) Yuan, X.; Pham, A. N.; Xing, G.; Rose, A. L.; Waite, T. D. Effects of pH, Chloride, and Bicarbonate on Cu(I) Oxidation Kinetics at Circumneutral pH. Environmental Science & Technology 2012, 46 (3), 1527-1535. DOI: 10.1021/es203394k.
(38) Moffett, J. W.; Zika, R. G. Reaction kinetics of hydrogen peroxide with copper and iron in seawater. Environmental Science & Technology 1987, 21 (8), 804-810. DOI: 10.1021/es00162a012.
(39) Bielski, B. H. J.; Cabelli, D. E.; Arudi, R. L.; Ross, A. B. Reactivity of HO2/O-2 Radicals in Aqueous Solution. Journal of Physical and Chemical Reference Data 1985, 14 (4), 1041-1100. DOI: 10.1063/1.555739 (acccessed 2023/03/19).
(40) Zafiriou, O. C.; Voelker, B. M.; Sedlak, D. L. Chemistry of the Superoxide Radical (O2-) in Seawater: Reactions with Inorganic Copper Complexes. The Journal of Physical Chemistry A 1998, 102 (28), 5693-5700. DOI: 10.1021/jp980709g.
(41) He, D.; Jones, A. M.; Garg, S.; Pham, A. N.; Waite, T. D. Silver Nanoparticle-Reactive Oxygen Species Interactions: Application of a Charging-Discharging Model. The Journal of Physical Chemistry C 2011, 115 (13), 5461-5468. DOI: 10.1021/jp111275a.
(42) Kondo, K.; Nakamura, T.; Okamoto, N. Correlation between Cu (I)-complexes and filling of via cross sections by copper electrodeposition. Journal of Applied Electrochemistry 2009, 39 (10), 1789-1795. DOI: 10.1007/s10800-009-9878-2.

We claim:

1. A device with microbiocidal or microbiostatic properties, comprising:
a device body that contains a first external surface and a second external surface that resides opposite said first external surface, and
an at least one antimicrobial metal component in electrical relationship to said first external surface and said second external surface.

2. The device in claim 1, wherein said at least one antimicrobial metal component is contained on, within or on and within said device body.

3. The device in claim 1, wherein a first electrically conductive lead is attached to said first external surface of said device body, and a second electrically conductive lead is attached to said second external surface of said device body which is electrically isolated from said first electrically conductive lead.

4. The device in claim 3, wherein said device further comprises an electrical power source connected to said first electrically conductive lead such that current flows from said first electrically conductive lead to said second electrically conductive lead through said device body.

5. The device in claim 4, wherein said electrical power source is provided by an internal battery, a capacitor or other AC or DC power source.

6. The device in claim 4, wherein said electrical power source causes the production of reactive oxidant species (ROS) and/or reactive chlorinated species (RCS) at the said antimicrobial metal component.

7. The device in claim 2, wherein said at least one antimicrobial metal component is in the form of a coating deposited on said first external surface of said device body and said coating is comprised of at least two metals.

8. The device in claim 2, wherein said at least one antimicrobial metal component is in the form of a metallic salt that forms a coating deposited on said first external surface of said device body.

9. The device in claim 7, wherein said coating ranges in thickness between 0.1×10−9 meters and 5×10−6 meters.

10. The device in claim 2, wherein said at least one antimicrobial metal component is contained within a metal component comprised of said at least one antimicrobial metal component in the range of 1%-100% by weight of the total composition of said metal component.

11. The device in claim 10, wherein said metal component contains an amount of said antimicrobial metal component in the range of about 1 nanogram to about 1 kilogram.

12. The device in claim 10, wherein said metal component may be provided as a substantially pure metal, an alloy, a composite, a mixture or a metal colloid.

13. The device in claim 2, wherein said at least one antimicrobial metal component is comprised of copper, silver, gold, copper, titanium, chromium, iron, nickel, zinc, palladium, platinum, tungsten, cobalt, vanadium or salt thereof.

14. The device in claim 13, wherein the at least one antimicrobial metal component includes a plurality of three-dimensional microparticles which can vary in a range of 10 μm to 5000 μm in width.

15. The device in claim 2, wherein said at least one antimicrobial metal component comprises a plurality of nanowires and said plurality of nanowires are monometallic and/or bimetallic that are deposited on said device body.

16. The device in claim 15, wherein said plurality of nanowires are composed of monometallic or multi-metallic materials that include copper, silver, gold, copper, titanium, chromium, iron, nickel, zinc, palladium, platinum, tungsten, cobalt and vanadium.

17. The device in claim 15, wherein a diameter of said plurality of nanowires ranges from 1 nm-5000 μm.

18. The device in claim 2, wherein said antimicrobial metal component is comprised of a conductive graphite coating applied to said first external surface.

19. A method for creating a microbiocidal or microbiostatic environment against one or more microorganisms, comprising:
providing a device body with an at least one antimicrobial metal component on, within or on and within said device body, and
wherein said plurality of antimicrobial metal components comprise of at least two different antimicrobial metals, and
applying an electrical current to said plurality of antimicrobial metal component to generate Reactive Oxygen Species or Reactive Chlorinated Species that possess antimicrobial properties.

20. The method in claim 19, wherein the microorganisms are deactivated with an applied potential of 0V to 11V.

21. The method in claim 20, wherein the microorganisms that are deactivated are lentivirus and/or Aspergillus.
